(19) United States
(12) Patent Application Publication
(10) Pub. No.: US 2012/0195954 A1
(43) Pub. Date: Aug. 2, 2012

(54) Method of Reducing Appetite

(75) Inventor: James H. Maynard, Morristown, NJ (US)

(73) Assignee: ROXITICUS HEALTH PRODUCTS, LLC, Morristown, NJ (US)

(21) Appl. No.: 13/363,672

(22) Filed: Feb. 1, 2012

Related U.S. Application Data
(60) Provisional application No. 61/438,699, filed on Feb. 2, 2011.

(51) Int. Cl. A61K 36/534; A61K 9/70; A61K 9/68; A61P 3/00; A61P 25/04

(52) U.S. Cl. 424/443; 424/747; 424/48

(57) ABSTRACT

Provided, among other things, is a method of reducing dietary intake comprising:

eating a portion of food; and immediately thereafter applying to the mouth a long-lasting, taste-blocking agent (strongly flavored agent and/or analgesic) in an appetite reducing effective amount.

This application claims priority of Ser. No. 61/438,699 filed Feb. 2, 2011, which is incorporated herein in its entirety.

The present invention relates to methods and tools for reducing appetite, and reducing weight.

To reduce appetite, Jackson, US 2005/0037031 has suggested blocking the sense of smell. One document found on the internet, by Swanner, suggests using an altoid to avoid eating. (The extent to which this document is or was indexed sufficiently to be a publication is unclear.) Among the things that Swanner does not describe are (a) a method of partial meal avoidance and (b) a sufficiently long-lasting, strongly flavored agent.

We eat for many reasons, including to sustain life, to be social, to feel full, to feel satisfied, to relieve depression, but mostly because we like the way food tastes. The taste stimulus is one of nature's primary motivators of human behavior. Nature created taste as a very strong inducement for people to put things in their mouths and swallow them, in order to supply the energy and nutrition necessary for life. However, in the modern context, the abundance of food and drink in developed nations and the additives in many foods and drinks, transforms the taste stimulus from a healthy survival motivator for individuals into a significant national health threat.

The medical cost and burden to society of obesity is enormous. The risks of obesity include heart disease, diabetes, peripheral vascular disease, cancer, strokes, arthritis, joint disease, depression, increased surgical risks, hip fractures, knee replacements, sleep apnea, high cholesterol, premature death, and much more. Obesity is bad for our health, bad for our society, and is as much a disease as are high blood pressure and pneumonia.

A number of diets focus on the symptom of obesity; excessive caloric intake. The methods and tools of the invention address the cause of excessive caloric intake; which is taste. Thus, the invention provides methods and tools that addresses the real problem—the taste of the foods we love to eat.

The flavor masking product and avoidance diet method described herein provides critical willpower support by reducing the motivational influence of taste on the decision making process for when, what, and how much to eat. This results in an increase in self-control and reasonable eating decisions based on rational health concepts; versus subconscious drives, emotional needs, or impulsive-indulgent eating behavior that turns nutrients into toxins due to their consumption in excessive quantity.

In one embodiment of the invention, the taste-masking effect is created by or supplemented with an analgesic agent. Analgesic has been found to deaden taste.

### SUMMARY OF THE INVENTION

Provided is a method of reducing dietary intake comprising: eating a portion of food; and immediately thereafter applying to the mouth a long-lasting, taste-blocking agent in an appetite reducing effective amount. the taste-blocking agent can be applied in a form adapted to release the appetite reducing effective amount in the mouth for ten minutes or more. An adhesive portion of a tab can be, or in the mouth can transition to, a waxy or gel-like form. The tab can be a mouth-adherent film or tablet.

Further provided is an oral delivery form that is mucoadhesive and (a) the taste-blocking agent is packaged so that a diffusion-limiting membrane allows but limits the rate of diffusion of taste-blocking agent therethrough into the mouth, wherein for a period of time the diffusing amount is an effective amount, or (b) wherein an effective amount of the taste-blocking agent is comprised within the adhesive layer of a patch, and wherein the patch has a backing layer adapted to allow diffusion of the taste-blocking agent therethrough.

Also provided as one option is a delivery form comprising at least (i) a delivery formulation that, if unchewed, delivers an effective amount of the taste-blocking agent to the mouth for an appropriate amount of time and (ii), intimately associated with but distinct from the delivery formulation, a chew retarding formulation that, chewed, delivers a strongly unpleasant flavor but that, unchewed, sufficiently retains the unpleasant flavor.

Further provided is an oral delivery form comprising effective amount of an analgesic and an effective amount of a strongly-flavored agent, wherein the amount of strongly-flavored agent is a super-flavoring amount.

### BRIEF DESCRIPTION OF THE DRAWINGS

FIG. 1 shows an embodiment in which there are modules of bad flavor.

### DETAILED DESCRIPTION OF THE INVENTION

The taste-blocking tools of the invention allow a person to control their eating by removing a primary inducement to eat: flavor. The user can elect to avoid consuming food or drink altogether, or to eat and/or drink a portion and then take the flavor agent to help limit further eating overeating or drinking. For example, in a restaurant or at a communal meal there is often a greater selection or portion of food than would be compatible with the portion control one seeks—yet the natural impulse is to continue to eat while flavorful food is available. Using the taste-blocking tools of the invention reduces the impulse, and psychologically further imprints the idea that one will not eat beyond the prescribed portion.

One specific tactic is portion control, which is limiting the quantity of any food or drink. Even healthful food and drink choices may nevertheless become unhealthy and add weight if overeaten. At times it may be necessary, as a part of a weight loss program, to limit even healthful food and drink which would normally be eaten in response to legitimate hunger.

A second tactic is control of binge eating and snacking, which is eating food that is not healthful or necessary and is for example motivated by recreational, emotional or “situational” stimuli rather than hunger.

In any situation, including portion control and binge/snack control situations, the invention helps the user to achieve diet goals by supporting the decision and willpower to stop eating when appropriate. The product does this by the process of removing, altering or minimizing flavor, which is a strong inducement to eat; and especially to eat the wrong things, or to over-eat. As an example, one can apply an appropriate strongly flavored agent to the mouth, and then smell or taste a food material that is usually highly enticing to the user. In the typical situation, the subject will then find the smell and/or taste no longer enticing. Though a primary effect is believed to be on gustatory taste receptors, it is believed that the brain compiles the taste information with olfactory inputs, which can alter perception of otherwise enticing odors.

In one embodiment, the methods and tools of the invention can be used to avoid an entire meal. When a user wants to skip a meal completely use the strongly flavored agent before your meal begins. If necessary given the form of the strongly flavored agent, the user may reapply the taste-masking agent as needed until the urge to eat has dissipated or until the user leaves the environment where food is available. If the urge returns or the environment is unfavorable to dieting reapply the strongly flavored agent.

In an important embodiment, the methods and tools of the invention can be used to limit the portion of food and/or drink taken at a meal setting. This process allows one to eat whatever he or she wants, thereby enjoying the taste, smells and pleasures associated with any particular food—but to limit total caloric consumption by using the taste agent when the user has had a diet-appropriate amount or at a predetermined time, e.g., half a meal. This allows one to enjoy the benefits of an unstructured, social meal, while limiting the inducements inherent in such a setting to overeat.

In another embodiment of the invention, the methods and tools of the invention can be used to avoid or minimize certain types of situations where historically one has overeaten. One can use either full or partial strategies for such situational avoidance. For example, one can choose to avoid the whole cocktail hour, or, one could use the taste agent to partially avoid the situation, namely to eat some food and then use the strongly flavored agent. Examples of such situations that can be avoided or minimized are cocktail hours, super bowl parties, bar food, the desert course of a meal, late night snacking, munchies, eating for emotional reasons, celebrations, junk food hangouts, and the like.

In still another embodiment, the methods and tools of the invention can be used to avoid or minimize alcohol consumption. Avoiding overdrinking is not only good for losing weight but also helps avoiding driving while intoxicated which can pose serious health and legal risks. Alcohol is fattening and it can harm the liver, and further it decreases inhibitions making it more likely for one to overeat or further over drink. The invention can be used either for complete avoidance, e.g., before one begins to drink, or to limit consumption, for example, after 1 or 2 drinks.

The taste-blocking tools typically use delivery form (“tab”) that is adapted to reside in the mouth for a sustained period. A tab will generally have enough strongly flavored agent and, if present, analgesic for maintaining food avoidance for the sustained period, or the appropriate integer fraction thereof if more than one tab (such as two or three) is to be used with each administration. The tab contains a strongly flavored agent and/or an analgesic agent, i.e, a “taste-blocking agent.”

### Strongly Flavored Agents

One exemplary strongly flavored agent is the extract/oil of peppermint, from Mentha×piperita. The agent is typically applied in a form that is stronger than found in many breath mints, and in a form that is adapted, if used per instruction, to last longer than a breath mint. Another exemplary strongly flavored agent is the extract/oil of spearmint, from Mentha spicata. Another exemplary strongly flavored agent is wintergreen oil, e.g., methy salicylate or extract/oil of Gaultheria procumbens. A number of plants of the genus Mentha are believed to provide useful oils/extracts. Capsaicin and zinc (such as without limitation zinc gluconate, zinc sulfate or zinc acetate) provide further strongly flavored agents. In one embodiment, the metallic flavor of zinc is deemed less than sufficiently strongly flavored—in which case zinc can be used as supplementary agent for blocking taste perception.

The flavor is generally adapted to be pleasant, or at least not unpleasant. In one embodiment, the idea is to not detract from the pleasant taste sensations of a meal, while nonetheless altering flavor appreciation sufficiently that the desire to take more food is reduced. However, in some embodiments, the flavor is unpleasant.

Strongly flavored agents are preferably already established as GRAS (generally recognized as safe) by the US Food and Drug Administration, and, if relevant, in amounts deemed safe pursuant to the Cumulative Estimated Daily Intake/Acceptable Daily Intake Database maintained by the US Food and Drug Administration.

Some of the strongly flavored agents are believed to contain agents having known, mild therapeutic effects. For example, peppermint oil contains menthol (2-isopropyl-5-methylcyclohexanol, especially its 1R,2S,5R form), that is known to trigger a cooling sensation (believed to be via the transient receptor potential cation channel, subfamily M, member 8, also known as TRPM8), and to be an analgesic (believed to be mediated through activation of κ-opioid receptors). Without being bound by theory, effects via TRPM8 or opioid receptors may contribute to appropriate taste altering effects.

The amount of strongly flavored agent present in a typical tab will depend on the particular strongly flavored agent. Where peppermint is the strongly flavored agent, the amount can be, for example, about the amount in an Altoid mint or more. Amounts may be adjusted dependent on a given segment of the population for which the tab is formulated. For example, certain populations may be more sensitive to strongly flavored agents, and the amount can be adjusted downwards for tabs intended for usage with these populations. Or, certain populations may be less sensitive to strongly flavored agents, and the amount can be adjusted upwards for tabs intended for usage with these populations. Populations that can be targeted include for example children (optionally of various age groups), adults of various age groups, those with various medical conditions, and the like.

### Analgesic Agents

Analgesics can be, for example, procaine benzocaine, chloroprocaine, cocaine, cyclomethycaine, dimethocaine/larocaine, piperocaine, propoxycaine, procaine/novocaine, proparacaine, tetracaine/amethocaine, lidocaine, articaine, bupivacaine, cinchocaine/dibucaine, etidocaine, levobupivacaine, lidocaine/lignocaine, mepivacaine, prilocaine, ropivacaine, trimecaine, or the like, and naturally derived local anesthetics, such as saxitoxin, tetrodotoxin, menthol, eugenol, kava, or the like. Combinations may be used. The formulation may contain a portion or all free base form, or be in salt form (e.g., acid addition salt). Counter ions will be pharmaceutically acceptable such ions. Where the strongly flavored agent is an analgesic, in this embodiment there is a second analgesic in the tab.

The amount of analgesic agent present in a typical tab will depend on the particular analgesic agent. Where benzocaine is the analgesic agent, the amount can be, for example, comparable to that in a pea-size dollop of 20% wt gel, or more. Amounts can be adjusted based on particular populations.

Without being bound by theory, it is believed that analgesics work by blocking the action of taste receptors. Without being bound by theory, it is believed that analgesics that act on the tongue are particularly useful. Without being bound by theory, it is believed that transmucosally deliverable analgesics are particularly useful. Without being bound by theory, it is believed that an amount of analgesic that deadens pin-prick sensation in the mouth will be an appetite reducing effective amount (for the period of such reduced mouth feel).

### Additional Agents

Other diet promoting agents can be added to the tabs, such as gymnema, a herbal agent that is believed to block sugar taste receptors and ease sugar craving.

### Compositions/Formulations

In certain embodiments, the taste-blocking agents are applied to the mouth in particular formulations. For example, in a tab that is a mouth-adherent film or tablet. Mouth-adherent compositions are known in the art and include, for example, the denture liners described in US 20070196787, 20080293015, 20050228066 and 20040028930, or the denture liner found in Polygrip™ Comfort Seal™ Strips, or the mouth deodorizing films described in U.S. Pat. No. 7,407,669. The films of the '669 patent are believed to generally dissolve too fast to be preferred in the present invention, but can be modified with more hydrophobic polymers and with waxes such as used in the denture liners to provide a more preferred residence time in the mouth. Though not a required feature of the invention, these films can be non-adhesive in dry form, but become adhesive as they wet in the mouth. This feature makes them relatively easy to handle. The commercial denture liner, Polygrip™ Comfort Seal™ Strips, is believed to comprise PEG-90M (high molecular weight polyol), microcrystalline wax, polybutene and cellulose gum in the adhesive, with the adhesive slab sandwiched between a thin, highly porous fabric that is infused or intimately associated with the adhesive.

In certain embodiments, the taste-blocking agents are applied in an adhesive form that is difficult to dislodge, so that the user is trained not to cheat on the regime by dislodging and swallowing. In certain embodiments, a waxy film form is used that cannot be easily dislodged, and which can be uncomfortable to dislodge. For example, films composed similar to Polygrip™ Comfort Seal™ Strips (with or without fabric) are believed to be difficult to dislodge in this fashion.

Tablets containing the taste-blocking agents can be coated with or compressed against an adhesive composition. If needed, the mouth adherent surface can include the thin, highly porous fabric mentioned above, or be packaged with a release liner (as is known in the art).

Any number of taste-blocking agents are believed to be compatible with film-forming adhesive compositions. To the extent that the polymer/plasticizer components of a film need to be modified in view of the taste-blocking agents, many of which it is believed will act as plasticizers, such modification is within the skill of the art.

The taste-blocking agents can be formulated in a chewing gum as is know in the art. The taste-blocking agents can be compounded with time-release components such as polymers (including hydrophobic polymers or a mixture of hydrophobic and hydrophilic polymers), for example such that time release nodules are dispersed through the gum. In some embodiments, mastication of nodules speeds release from those nodules. In certain embodiments, the nodules have individual time release coatings, such as formed by a fluidized bed coating process, or by another process known in the art.

Patch designs are well known in the art, as illustrated in http://www.uspharmacist.com/index.asp?show=article&page=8_{—}1061.htm; and http://www.pharmainfo.net/reviews/transdermal-drug-delivery-systems-review (both as downloaded from the internet Dec. 16, 2008). Patches tend to fall in two categories. Most prevalent are those in which the adhesive (“matrix”) serves as the primary reservoir for the agent to be released. The other category is for patches that contain a separate layer or housing that provides the primary reservoir. Contrary to many traditional transdermal patches, but well within the skill in the art, the backing layer used in the patch will generally be selected to allow diffusion of the taste-blocking agent from the reservoir and through the backing layer. The extent of transmission through the backing layer can be selected to provide an appropriate rate of release of the taste-blocking agent. In certain embodiments, transmission through the backing layer is a minor source of distributing taste-blocking agent into the mouth, with the boundaries of the patch or the tissue adherent side providing all or the bulk of transmission.

Traditional patches, that are intended to deliver bioactive into the underlying strata, give rise to problematic issues that do not pertain in the present context. Here, in certain embodiments, one needs not be concerned with the extensive experimentation needed to find compositions and delivery enhancers that deliver to the underlying strata, as delivery is instead aimed at a medium that allows relatively free diffusion.

The taste-blocking agent can be encapsulated in a membrane that allows diffusion of the taste-blocking agent therethrough, but limits the rate of diffusion. For example, the membrane can be a microporous dialysis membrane, such as a cellulose-based dialysis membrane, or any other polymeric membrane that provides a useful, but rate limiting rate of diffusion of the taste-blocking agent therethrough. Such a membrane can be the backing layer of a patch.

In certain embodiments, the formulation of the taste-blocking agent is effective, if unchewed, to deliver an effective amount of the taste-blocking agent for a desired period of time. For example, the form could be a slowly dissolving tablet. Therefore, in certain embodiments it can be useful to train the user not to chew on the delivery form. One way to do so is to physically associate the taste-blocking agent formulation with another formulation including an agent whose taste is undesirable. For example, the bad flavor can be sandwiched between to non-dissolving or slowly dissolving layers, with the edges also blocked by the non-dissolving or slowly dissolving material. This sandwich can be adhered to, for example, a tablet containing the taste-blocking agent. Thus, if the user bits on the combined form, he or she releases the bad taste, and therefore a lesson to not chew again—while in many cases also receiving an effective, if less desirable, taste-blocking agent.

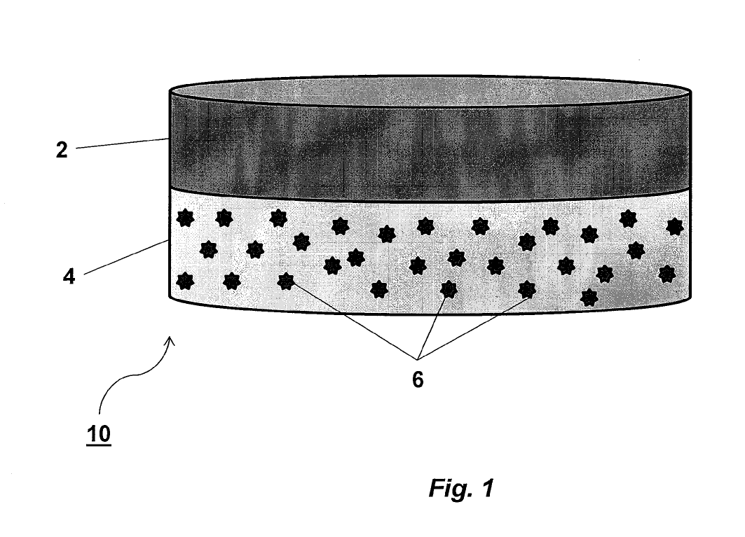

The bad flavor can also be encapsulated or otherwise formulated in nodules to provide for very slow to negligible release. Such nodules can be dispersed in the delivery form, such that chewing/biting release a sufficient bolus of bad flavor to remind the user not to chew, while preserving the bulk of the delivery form for continued use (with the more desired strong flavor thereafter reasserting itself). For example, tablet 10 in FIG. 1 has two layers, agent layer 2 contains the desired taste-blocking agent, while avoidance layer 4 contains modules 6 that contain bad flavor.

The tabs can be patches, gums, tablets, lozenges, hard candies, films, sprays, gel, and the like.

For most any of the above-described formulations, or for more simple formulations, the time of delivery of an effective amount of taste-blocking agent can be extended by instructing the user to apply the taste-blocking composition where there is delivery to the mouth, but less chance of chewing or other manipulation that might speed agent release. For example, the taste-blocking agent can be applied to the cleft between the lips and gums.

The form of the taste-blocking agent can be designed to, if used according to instruction, to release an appetite reducing effective amount of taste-blocking agent for 15 minutes or more, or 20 minutes or more, or 25 minutes or more, or 30 minutes or more, or 40 minutes or more. There may be a delay from application until an appetite reducing effective amount begins to be released, but this time is generally adapted not be so long as to impinge on the psychological commitment to avoid eating. For example, in preferred embodiments the delay is less than 2 minutes, more preferably less than one minute. Delivery forms with time release components can be combined with forms that provide more immediate release to assure this balance.

Appetite Reducing Effective Amount

An appetite reducing effective amount of a taste-blocking agent will be an amount that (a) reduces (e.g., inhibits alters, mitigates or masks) one or more gustatory perceptions of the food (e.g., of the five known, salty, sweet, sour, bitter and umamai (savory)) and (b) reduces appetite for the food. This effect may not hold for all foods, and may not hold in certain circumstances, such as notably elevated hunger, but shall be generally applicable. Optionally, the appetite reducing effective amount of a taste-blocking agent alters the perception of the odor of a food to generally reduce the savoriness of such food.

Mouth

Without limitation, the mouth includes the (i) the narrow cleft between the lips and gums and (ii) the oral cavity proper.

Oil-Based Flavor Extract

An oil-based flavor extract is a natural product flavor extract (or engineered mimic) in which a substantial portion of the flavoring agents are hydrophobic, i.e., favor partitioning in the organic phase of a octanol-water phase separation (molar basis). In certain embodiments, a substantial portion of the flavoring agents are favor partitioning in organic phase of a octanol-water phase separation by 5:1 or more, or 10:1 or more.

Oral Cavity Proper

The oral cavity proper is the space between the dental arches, limited posteriorly by the isthmus of the fauces (palatoglossal arch).

Super-Flavoring Amount

A super-flavoring amount of a strongly flavored agent is an amount that, as released by a tab, provides in the mouth for an operative period (e.g., 10 min. or more) a sustained amount of taste-masking effect as provided by a peppermint Altoid one minute after insertion into the mouth (and without chewing). The amount can be established by questionnaires using volunteer test subjects. In the various embodiments of the invention that use a strongly-flavored agent, the amount can be a super-flavoring amount.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of reducing dietary intake comprising:
eating a portion of food; and
immediately thereafter applying to the mouth a long-lasting, taste-blocking agent in an appetite reducing effective amount.

2. The method of claim 1, wherein the taste-blocking agent is applied in a form adapted to release the appetite reducing effective amount in the mouth for ten minutes or more.

3. The method of claim 2, wherein the taste-blocking agent is applied in a mouth-adherent film or tablet.

4. The method of claim 2, wherein an adhesive portion of a tab containing the taste-blocking agent is, or in the mouth transitions to, a waxy or gel-like form.

5. The method of claim 2, wherein the taste-blocking agent is applied as a chewing gum.

6. The method of claim 2, wherein the taste-blocking agent is applied as patch comprising a backing layer and an adhesive layer.

7. The method of claim 6, wherein an effective amount of the taste-blocking agent is comprised within the adhesive layer of the applied patch, and wherein the backing layer is adapted to allow diffusion of the taste-blocking agent therethrough.

8. The method of claim 6, wherein the applied patch comprises a reservoir layer and wherein an effective amount of the taste-blocking agent is comprised within the reservoir layer.

9. The method of claim 2, wherein the taste-blocking agent is applied with an amount thereof packaged so that a diffusion-limiting membrane allows but limits the rate of diffusion of taste-blocking agent therethrough into the mouth, wherein for a period of time the diffusing amount is an effective amount.

10. The method of claim 2, wherein the taste-blocking agent is applied in a delivery form comprising at least (i) a delivery formulation that, if unchewed, delivers an effective amount of the agent to the mouth for an appropriate amount of time and (ii), intimately associated with but distinct from the delivery formulation, a chew retarding formulation that, chewed, delivers a strongly unpleasant flavor but that, unchewed, sufficiently retains the unpleasant flavor.

11. The method of claim 10, wherein the unpleasant flavoring agent(s) are packaged in multiple nodules in the chew retarding formulation, so as to limit the amount of unpleasant flavor released on any given chew event.

12. The method of claim 1, wherein the taste-blocking agent is an oil-based flavor extract.

13. The method of claim 13, wherein the taste-blocking agent is substantially based on an extract from one or more plants of the genus Mentha.

14. The method of claim 1, wherein the taste-blocking agent is applied to the cleft between the lips and gums.

15. The method of claim 1, wherein the taste-blocking agent is an analgesic.

16. The method of claim 1, wherein the taste-blocking agent is a strongly-flavored agent.

17. The method of claim 1, wherein the taste-blocking agent is a mixture of a strongly-flavored agent and an analgesic.

18. An oral delivery form that: (i) is mucoadhesive and (a) the taste-blocking agent is packaged so that a diffusion-limiting membrane allows but limits the rate of diffusion of taste-blocking agent therethrough into the mouth, wherein for a period of time the diffusing amount is an effective amount, or (b) wherein an effective amount of the taste-blocking agent is comprised within the adhesive layer of a patch, and wherein the patch has a backing layer adapted to allow diffusion of the taste-blocking agent therethrough; or (ii) comprises an effective amount of an analgesic and an effective amount of a strongly-flavored agent, wherein the amount of strongly-flavored agent is a super-flavoring amount.

19. A delivery form comprising at least (i) a delivery formulation that, if unchewed, delivers an effective amount of the taste-blocking agent to the mouth for an appropriate amount of time and (ii), intimately associated with but distinct from the delivery formulation, a chew retarding formulation that, chewed, delivers a strongly unpleasant flavor but that, unchewed, sufficiently retains the unpleasant flavor.
